(19) United States
(12) Plant Patent Application Publication
(10) Pub. No.: US 2015/0096089 P1
(43) Pub. Date: Apr. 2, 2015

(54) Osteospermum plant named 'KLEOE13214'

(50) ;

(71) Applicant: Klemm+Sohn GmbH & Co. KG, Stuttgart (DE)

(72) Inventors: Nils Klemm, Stuttgart (DE); Silke Wagener, Leinfelden-Echterdingen (DE)

(73) Assignee: Klemm+Sohn GmbH & Co. KG, Stuttgart (DE)

(21) Appl. No.: 13/998,117

(22) Filed: Oct. 2, 2013

(51) Int. Cl. A01H 5/00

(52) U.S. Cl. PLT/360

(57) ABSTRACT

A new Osteospermum plant particularly distinguished by purple ray florets and green-yellow and purple disc florets, is disclosed.

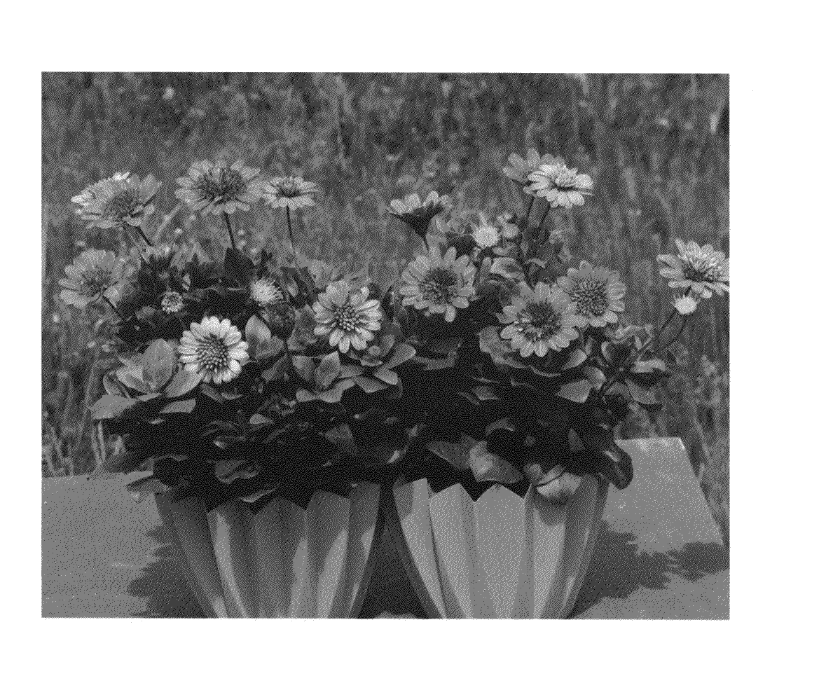

### GENUS AND SPECIES

Osteospermum ecklonis

### VARIETY DENOMINATION

‘KLEOE13214’

### BACKGROUND OF THE NEW PLANT

The present invention comprises a new and distinct variety of Osteospermum, botanically known as Osteospermum ecklonis, and hereinafter referred to by the variety name ‘KLEOE13214’. ‘KLEOE13214’ originated from a controlled cross conducted in summer 2009 in Nairobi, Kenya between the proprietary female Osteospermum variety ‘OE-2008-0390’ (unpatented), having light purple ray florets and purple disc, florets, and the proprietary male Osteospermum variety ‘OE-2007-0028’ (unpatented) having purple-orange ray florets and violet disc florets.

The seeds from the cross were sown and plants were grown in a greenhouse for evaluation, where an individual plant designated ‘KLEOE13214’ was selected from the group of plants in August 2010. In August 2010, ‘KLEOE13214’ was first vegetatively propagated by terminal vegetative cuttings. ‘KLEOE13214’ was found to reproduce true to type in successive generations of asexual propagation via terminal vegetative cuttings.

### CROSS REFERENCE TO RELATED APPLICATIONS

Plant Breeder's Rights for ‘KLEOE13214’ were applied for in Canada on Feb. 8, 2013, Application No. 13-7922. ‘KLEOE13214’ has not been made publicly available or sold more than one year prior to the filing date of this application.

### SUMMARY

The following are the most outstanding and distinguishing characteristics of this new variety when grown under normal horticultural practices in Stuttgart, Germany.

1. Purple ray florets; and

2. Green-yellow and purple disc florets.

### DESCRIPTION OF THE PHOTOGRAPH

This new Osteospermum plant is illustrated by the accompanying photograph which shows the plant's overall plant habit including form, foliage, and flowers. The photograph is of a four-month-old plant grown in a greenhouse in Stuttgart, Germany in May 2012. The colors shown are as true as can be reasonably obtained by conventional photographic procedures.

### DESCRIPTION OF THE NEW VARIETY

The following detailed descriptions set forth the distinctive characteristics of ‘KLEOE13214’. The data which define these characteristics were collected from asexual reproductions carried out in Stuttgart, Germany. Data was collected on four- to five-month-old plants grown in a greenhouse in Stuttgart, Germany in June 2013. Color references are to The R.H.S. Colour Chart of The Royal Horticultural Society of London (R.H.S.), 5^{th} edition (2007).
Classification:
   Family.—Asteraceae.
   Botanical.—Osteospermum ecklonis.
   Common.—Osteospermum.
   Designation.—‘KLEOE13214’.
   Parentage.—Female parent: The proprietary female Osteospermum variety ‘OE-2008-0390’ (unpatented) Male parent: The proprietary male Osteospermum variety ‘OE-2007-0028’ (unpatented).
Plant:
   Form and habit.—Herbaceous perennial but usually cultivated as an annual.
   Growth habit.—Upright.
   Branching habit.—Freely branching.
   Height (from top of soil, including inflorescence).—About 22.0 cm.
   Width (including inflorescences).—15.0 cm to 20.0 cm.
   Propagation.—Terminal cuttings.
   Time to produce a finished flowering plant.—14 to 16 weeks.
   Time to initiate and develop roots.—3 to 4 weeks.
   Root description.—Freely rooting.
Leaves:
   Arrangement.—Alternate.
   Shape.—Dentiform.
   Apex.—Obtuse.
   Base.—Acute.
   Margin.—Shallow serrate.
   Immature color.—Upper surface: RHS 143A Lower surface: RHS 143C.
   Mature color.—Upper surface: RHS 143A Lower surface: RHS 143B.
   Length.—About 6.5 cm.
   Width.—About 4.0 cm.
   Texture and appearance (upper and lower surfaces).—Leathery, shiny.
   Petioles.—Absent.
Stems:
   Total number of branches per plant (calculated at the base of the plant).—3 to 4.
   Length.—16.0 cm.
   Diameter.—About 0.4 cm.
   Internode length.—About 1.0 cm.
   Color.—RHS 141D.
   Texture.—Smooth.
   Anthocyanin.—Absent.
Flower buds:
   Shape.—Obovate.
   Length.—About 1.0 cm.
   Diameter.—About 0.8 cm.
   Color (at tight bud just before the ray florets unfold).—RHS 143B.
Inflorescence:
   Type.—Composite, single.
   Blooming habit.—Spring to autumn.
   Quantity of inflorescences per plant.—20 to 30.
   Lastingness of the inflorescences on the plant.—About 2 weeks.
   Fragrance.—Absent.
   Inflorescence diameter.—About 6.5 cm.
   Inflorescence depth.—2.0 cm.
   Disc diameter.—3.0 cm.
Disc floret:
   Quantity per inflorescence.—About 85.
   Shape.—Lanceolate.
   Tube color.—Closed: RHS 1A (outer surface at an immature state) Mature and inner surface: RHS 61A.
   Length.—About 1.5 cm.
   Diameter (at apex).—0.3 cm.
   Apex.—Obtuse.
   Apex color.—RHS 1A.
   Base.—Acute.
   Base color.—RHS 70B.
   Margin.—Dentate.
Ray floret:
   Quantity per inflorescence.—About 26.
   Shape.—Oblanceolate.
   Color.—Upper surface: RHS 71A and RHS 71D Lower surface: RHS 59B and RHS 58D.
   Length.—About 3.0 cm.
   Width.—About 0.8 cm.
   Apex.—Obtuse.
   Base.—Acute.
   Margin.—Entire.
   Texture.—Upper surface: Velvet, smooth Lower surface: Smooth.
Peduncle:
   Length.—About 6.0 to 7.0 cm.
   Diameter.—0.2 cm.
   Texture.—Rough.
   Color.—RHS 144B.
Phyllaries:
   Texture.—Rough.
   Color.—RHS 143B.
   Arrangement.—Single.
   Observed quantity per plant.—20 to 30.
   Shape.—Lanceolate.
   Color.—Upper surface: RHS 143B Lower surface: RHS 143A.
   Length.—About 0.6 cm.
   Width.—About 0.15 cm.
   Apex.—Acute narrowly.
   Base.—Rotundate.
   Margin.—Entire.
   Texture (both upper and lower surfaces).—Rough.
Reproductive organs:
   Androecium.—Location: On the disc floret Stamens: Quantity: Numerous Shape: Filamentous Filament length: 0.4 cm to 0.5 cm Filament diameter: Less than 0.05 cm Anther: Shape: Lanceolate Color: RHS 59A Length: 0.1 cm to 0.2 cm Diameter: Less than 0.05 cm Pollen: Color: RHS 21A Amount: Sparse.
   Gynoecium.—Location: On the base of the ray florets Pistils: Number: About 26 Length: 0.7 cm Diameter: Less than 0.05 cm Stigma: Color: RHS 59A Shape: Bipartite tapering Length: 0.1 cm Diameter: Less than 0.05 cm Style: Color: RHS 59A Length: 0.3.cm Diameter: 0.05 cm.
Fruit and seed set: None observed.
Disease and pest/insect resistance: No disease and pest/insect resistance observed.

### COMPARISON WITH PARENTAL LINES AND KNOWN VARIETY

‘KLEOE13214’ is a distinct variety of Osteospermum having purple ray florets and green-yellow and purple disc florets. ‘KLEOE13214’ is distinguished from its parents, the female parent ‘OE-2008-0390’ (unpatented) and the male parent, ‘OE-2007-0028’ (unpatented), as described in Table 1.

TABLE 1
Comparison with Parental Lines

| | | Female Parent | Male Parent |
|---|---|---|---|
| Characteristic | ‘KLEOE13214’ | ‘OE-2008-0390’ | ‘OE-2007-0028’ |
| Ray floret color | Purple | Light purple | Purple-orange |
| Disc floret color | Green-yellow and | Purple | Violet |
| | purple | | |

‘KLEOE13214’ is most similar to the commercial Osteospermum variety ‘KLEOE13214’ (unpatented). Differences between the varieties are described in Table 2.

TABLE 2
Comparison with Similar Variety

| Characteristic | ‘KLEOE13214’ | ‘KLEOE12198’ |
|---|---|---|
| Disc floret color | Green-yellow and purple | Purple-violet |

We claim:

1. A new and distinct variety of Osteospermum plant designated ‘KLEOE13214’ as shown and described herein.
